(19) United States
(12) Patent Application Publication
(10) Pub. No.: US 2025/0189854 A1
(43) Pub. Date: Jun. 12, 2025

(54) ORGANIC COMPOUND AND ELECTROCHROMIC ELEMENT

(71) Applicant: CANON KABUSHIKI KAISHA, Tokyo (JP)

(72) Inventor: SATOSHI IGAWA, Kanagawa (JP)

(21) Appl. No.: 18/975,654

(22) Filed: Dec. 10, 2024

(30) Foreign Application Priority Data
Dec. 12, 2023 (JP) 2023-209234
Jun. 3, 2024 (JP) 2024-090294

(51) Int. Cl. G02F 1/1516; B32B 17/10; C07D 471/04; C09K 9/02

(52) U.S. Cl. CPC G02F 1/1516; B32B 17/10036; B32B 17/10513; B32B 17/10779; C07D 471/04; C09K 9/02; C09K 2211/1018; G02F 2203/055

(57) ABSTRACT

An organic compound represented by the following formula (1) is provided:

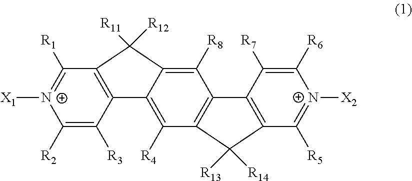

In general formula (1), X₁ and X₂ are each independently selected from the group consisting of substituted or unsubstituted alkyl groups, substituted or unsubstituted aryl groups, and substituted or unsubstituted aralkyl groups. R₁ to R₈ are each independently selected from the group consisting of a hydrogen atom, a deuterium atom, halogen atoms, substituted or unsubstituted alkyl groups, substituted or unsubstituted alkoxy groups, and substituted or unsubstituted aryl groups. R₁₁ to R₁₄ are each independently selected from the group consisting of a hydrogen atom, substituted or unsubstituted alkyl groups, and substituted or unsubstituted aryl groups.

### BACKGROUND

### Technical Field

The present disclosure relates to an organic compound and an electrochromic element using the organic compound.

### Description of the Related Art

An electrochromic (hereinafter also abbreviated to EC) element includes a pair of electrodes and an EC layer disposed between the pair of electrodes. The EC element can adjust the amount of light passing through the EC layer when voltage is applied to the pair of electrodes. In other words, the EC element can control its light transmittance.

EC materials are substances whose optical absorption properties (e.g., coloring state and light transmittance) are varied by electrochemical redox reaction. Various materials, including inorganic, low-molecular-weight organic, and polymer materials, are known as EC materials.

Such EC materials have been used in EC elements applied to dimming mirrors in automobiles, electronic papers, and other devices. These devices take advantage of the characteristic of EC materials that a variety of color tones can be displayed depending on the selection of the material. Developing materials for various color tones is desired for using EC elements. For example, for application to full-color display devices or the like, materials to color cyan, magenta, or yellow are used. For a wider range of applications, coloring materials for various colors and tones are required.

Japanese Patent Laid-Open No. 2020-152708 (hereinafter abbreviated to PTL 1) discloses a cathodic EC compound 1-a:

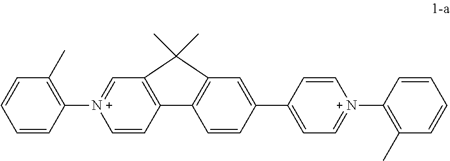 [SMILES: Cc1ccccc1-[n+]1ccc(-c2ccc3c(c2)C(C)(C)c2c[n+](-c4ccccc4C)ccc2-3)cc1]

However, the compound disclosed in PTL 1 has room for improvement in terms of stability in the first reduced state.

### SUMMARY

The present disclosure provides an organic compound that is more stable in terms of absorbable light wavelength.

The organic compound is represented by general formula (1):

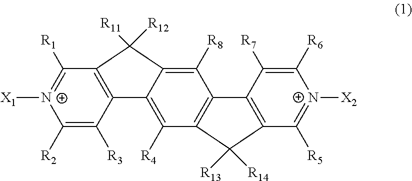

In general formula (1), X₁ and X₂ are each independently selected from the group consisting of substituted or unsubstituted alkyl groups, substituted or unsubstituted aryl groups, and substituted or unsubstituted aralkyl groups.

R₁ to R₈ are each independently selected from the group consisting of a hydrogen atom, a deuterium atom, halogen atoms, substituted or unsubstituted alkyl groups, substituted or unsubstituted alkoxy groups, and substituted or unsubstituted aryl groups.

R₁₁ to R₁₄ are each independently selected from the group consisting of a hydrogen atom, substituted or unsubstituted alkyl groups, and substituted or unsubstituted aryl groups.

Further features of the present disclosure will become apparent from the following description of exemplary embodiments with reference to the attached drawings.

### BRIEF DESCRIPTION OF THE DRAWINGS

FIG. 1 is a schematic sectional view of an example of an electrochromic element according to an embodiment.

FIG. 2 is a schematic diagram of an example of a drive unit including an electrochromic element according to an embodiment.

FIG. 3A is a schematic diagram of an example of an imaging device in which an optical filter is disposed in a lens unit.

FIG. 3B is a schematic diagram of an example of an imaging device in which an optical filter is disposed in an imaging unit.

FIG. 4A is a schematic diagram of an example of a window using an EC element according to an embodiment.

FIG. 4B is a schematic sectional view taken along line IVB-IVB in FIG. 4A.

FIG. 5 is a plot of UV-visible absorption spectra of Example Compound A-6 in a coloring state and an achromatizing state.

FIG. 6 is a plot of UV-visible absorption spectra of Example Compound A-41 in a coloring state and an achromatizing state.

FIG. 7 is a plot of UV-visible absorption spectra of Example Compound A-18 in a coloring state and an achromatizing state.

### DESCRIPTION OF THE EMBODIMENTS

In the description herein, halogen atoms used herein include, but are not limited to, fluorine, chlorine, bromine, and iodine atoms.

The alkyl groups may have 1 to 20 carbon atoms, for example, 1 to 12 carbon atoms or 1 to 10 carbon atoms. The alkyl groups may be linear, branched, or cyclic. Examples of alkyl groups include, but are not limited to, methyl, ethyl, n-propyl, isopropyl, n-butyl, tert-butyl, sec-butyl, octyl, cyclohexyl, 1-adamantyl, and 2-adamantyl. Substituents that the alkyl groups may have include, but are not limited to, halogen atoms, ester groups, and a cyano group. Also, a halogen atom, for example, a fluorine atom, may substitute for a hydrogen atom of the alkyl group. An ester or a cyano group may substitute for a hydrogen atom of the alkyl group.

The aryl groups may have 6 to 20 carbon atoms, for example, 6 to 10 carbon atoms. Examples include, but are not limited to, phenyl, biphenyl, terphenyl, fluorenyl, naphthyl, fluoranthenyl, anthryl, phenanthryl, pyrenyl, tetracenyl, pentacenyl, triphenylenyl, and perylenyl. The aryl groups may have at least one of a halogen atom, an alkyl group with 1 to 8 carbon atoms, and an alkoxy group with 1 to 8 carbon atoms as a substituent. Also, a halogen atom, for example, a fluorine atom, may substitute for a hydrogen atom of the alkyl or alkoxy group.

The alkoxy groups may have 1 to 10 carbon atoms, for example, 1 to 4 carbon atoms. Examples of such alkoxy groups include, but are not limited to, methoxy, ethoxy, propoxy, isopropoxy, butoxy, tert-butoxy, 2-ethyl-octyloxy, and benzyloxy.

Examples of aryloxy groups include, but are not limited to, phenoxy.

The aralkyl groups may have 7 to 20 carbon atoms, for example, 7 to 10 carbon atoms. Examples include, but are not limited to, benzyl and phenethyl. The aralkyl groups may have one or more substituents, specifically, an alkyl group having 1 to 8 carbon atoms or an alkoxy group having 1 to 8 carbon atoms. A halogen atom, for example, a fluorine atom, may substitute for a hydrogen atom of the alkyl or alkoxy group.

Examples of ester groups include, but are not limited to, carboxylate ester groups, sulfonate ester groups, and phosphonate ester groups.

The term cathodic EC compound used herein refers to an organic compound that exhibits a color when reduced. The term anodic EC compound refers to an organic compound that exhibits a color when oxidized.

Also, in the description herein, “coloring” means that the transmittance at a specific wavelength decreases.

An organic compound that exhibits a color when reduced refers to an organic compound whose visible light transmittance in reduction is lower than in oxidation. In contrast, an organic compound that exhibits a color when oxidized refers to an organic compound whose visible light transmittance in oxidation is lower than in reduction. The transmittance has to vary at any of the wavelengths in the visible region but need not vary throughout the entire visible region.

### (1) Organic Compound

The organic compound disclosed herein will first be described.

The organic compound disclosed herein (hereinafter also referred to as the present organic compound) is represented by the following general formula (1) and is electrochromic. Accordingly, the present organic compound may also be referred to as the EC compound. The present organic compound colors when reduced.

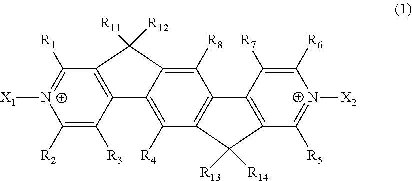

In general formula (1), X₁ and X₂ are each independently selected from the group consisting of substituted or unsubstituted alkyl groups, substituted or unsubstituted aryl groups, and substituted or unsubstituted aralkyl groups.

X₁ and X₂ may be alkyl groups with 1 to 12 carbon atoms, aryl groups with 6 to 10 carbon atoms, or aralkyl groups with 7 to 10 carbon atoms, for example, alkyl groups with 1 to 10 carbon atoms or aryl groups with 6 to 10 carbon atoms, and, in some embodiments, may be alkyl groups with 1 to 7 carbon atoms or aryl groups with 6 to 10 carbon atoms, for example, heptyl or phenyl, particularly heptyl.

X₁ and X₂ may have the same or different structures, but in some embodiments, they may have the same structure from the viewpoint of ease of synthesis.

When X₁ and X₂ are alkyl groups, the alkyl groups may have adsorbable groups or their acid ester groups to be adsorbed on an electrode. The electrode may be porous. Specific examples of adsorbable groups and their acid ester groups include carboxyl and carboxylate ester groups, sulfonic and sulfonate ester groups, phosphonic and phosphonate ester groups, and trialkoxysilyl groups. X₁ and X₂ having adsorbable groups or their acid ester groups may include carboxyl, carboxylate ester, sulfonic, phosphonic, or phosphonate ester groups and, in some embodiments, may include carboxyl, sulfonic, phosphonic, or phosphonate ester groups.

In an embodiment, an alkyl group of the present organic compound may have a polymerizable functional group at an end thereof to make the compound polymeric. Specific examples of the polymerizable functional group include acrylic, methacrylic, and hydroxy groups. Also, the alkyl group may have an ionic group, such as pyridinium or quinolinium, at an end thereof to increase the solubility in organic solvent.

When X₁ and X₂ are aralkyl or aryl groups having an alkyl or alkoxy group as a substituent, the substituent may have, at an end thereof, an adsorbable group or its acid ester group to be adsorbed on the electrode or a polymerizable functional group to make the compound polymeric. Also, the substituent may have an ionic group to increase the solubility in organic solvent. Specific examples of the adsorbable group and its acid ester group and the ionic group are the same as cited above. X₁ and X₂ having adsorbable groups, their acid ester groups, or ionic groups may have carboxyl, carboxylate ester, sulfonic, phosphonic, phosphonate ester, or pyridinium groups and, in some embodiments, may have carboxyl, sulfonic, phosphonic, phosphonate ester, or pyridinium groups.

R₁ to R₈ are each independently selected from the group consisting of a hydrogen atom, a deuterium atom, halogen atoms, substituted or unsubstituted alkyl groups, substituted or unsubstituted alkoxy groups, and substituted or unsubstituted aryl groups.

R₁ to R₈ each may be a hydrogen atom, a deuterium atom, a halogen atom, an alkyl group with 1 to 4 carbon atoms, an aryl group with 6 to 10 carbon atoms, or an alkoxy group with 1 to 4 carbon atoms, for example, a hydrogen, deuterium, or fluorine atom or a methyl, methoxy, or phenyl group, and in some embodiments, may be a hydrogen or fluorine atom or a methyl, methoxy, or phenyl group, particularly a hydrogen atom.

R₁₁ to R₁₄ are each independently selected from the group consisting of a hydrogen atom, substituted or unsubstituted alkyl groups, and substituted or unsubstituted aryl groups.

R₁₁ to R₁₄ each may be a hydrogen atom, a deuterium atom, an alkyl group with 1 to 4 carbon atoms, and an aryl group with 6 to 10 carbon atoms or may be a hydrogen atom, a deuterium atom, an alkyl group with 1 to 4 carbon atoms, or a phenyl group, particularly a hydrogen atom or a methyl group.

In an embodiment, the present organic compound may contain monovalent anions A₁⁻ and A₂⁻ as counter ions. A₁⁻ and A₂⁻ are each independently selected from anions such as PF₆⁻, ClO₄⁻, BF₄⁻, AsF₆⁻, SbF₆⁻, CF₃SO₃⁻, and (CF₃SO₂)₂N⁻ and halogen anions, such as Br⁻, Cl⁻, and I⁻. For example, A₁⁻ and A2⁻ may be Cl⁻, Br⁻, I⁻, BF₄⁻, PF₆⁻, ClO₄⁻, CF₃SO₃⁻, or (CF₃SO₂)₂N⁻.

A₁⁻ and A₂⁻ may have the same or different structures, but in some embodiments, they may have the same structure from the viewpoint of ease of synthesis.

In other words, the organic compound represented by general formula (1) may be represented by general formula (2):

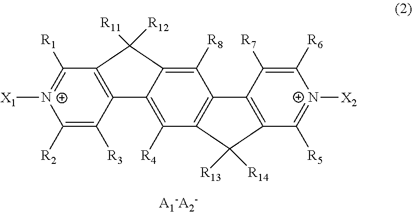

In general formula (2), R₁ to R₈, R₁₁ to R₁₄, X₁ and X₂, and A₁⁻ and A₂⁻ have the same structures as those of general formula (1).

The organic compound represented by general formula (1) has the following features:
(1-1) Having a stable structure in the first reduced state; and
(1-2) Being durable because of the fused ring skeleton.

These features will now be described.

### (1-1) Having a Stable Structure in the First Reduced State

The organic compound disclosed herein, which is represented by general formula (1), has a more stable structure than the organic compound disclosed in PTL 1, in the first reduced state. More specifically, the difference between the first and second reduction potentials of the present organic compound is larger than that of the organic compound disclosed in PTL 1.

The effect of the large difference between the first and second reduction potentials will now be described. EC compounds vary in their absorption wavelength through redox reactions, thereby being colored or achromatized.

Some EC compounds have different absorption wavelengths between the first reduced state (one-electron reduced state) and the second reduced state (two-electron reduced state). In this instance, when the difference between the first and second reduction potentials is small, local changes in potential or the like increase the possibility of a mixture of the first and second reduced states in the system. As a result, the EC compound becomes likely to absorb light with unintended wavelengths, becoming unstable in terms of absorbable light wavelength. In contrast, when the difference between the first and second reduction potentials is large, the system is less likely to contain a mixture of the first and second reduced states. As a result, the EC compound becomes more stable in terms of absorbable light wavelength. Thus, the difference between the first and second reduction potentials may be large. Specifically, the difference between the first and second reduction potentials may be larger than 0.12 V and is, in some embodiments, 0.15 V or more, or 0.17 V or more, particularly 0.20 V or more.

The difference between the first and second reduction potentials can be measured by, for example, cyclic voltammetry (CV). More specifically, a solvent, and a 0.1 mol/L electrolyte in the solvent, and 0.5 mmol/L of the EC compound in the solvent are dissolved to prepare a solution. The resulting solution is subjected to CV measurement in a three-electrode cell with a working electrode, a counter electrode, and a reference electrode, in which E_{1/2} of ferrocene as the reference material is set as 0 V, and the largest reduction potential of the reduction peaks of the EC compound is defined as the first reduction potential, and the second largest reduction potential is defined as the second reduction potential. Thus, the difference between the first and second reduction potentials can be obtained. E_{1/2}, which is called half-wave potential, is the midpoint between the oxidation potential and the re-reduction potential or the midpoint between the reduction potential and the re-oxidation potential. Also, reduction potentials are typically expressed as negative values. Hence, a high reduction potential refers to a small absolute value of the reduction potential.

The present organic compound has a larger difference between the first and second reduction potentials than the organic compound disclosed in PTL 1 and is, accordingly, less likely to absorb light with unintended wavelengths as an EC compound. Also, when the difference between the first and second reduction potentials of the present organic compound is 0.12 V or more, the EC compound does not easily absorb light with unintended wavelengths.

### (1-2) Being Durable Because of the Fused Ring Skeleton

The present organic compound is more durable than compound 1-a because it has a fused ring skeleton.

The radical cations of x-conjugated molecules are stabilized by resonance, and the π-conjugated portion is expected to become flat. Compound 1-a, which has a skeleton in which an azafluorene ring is bound to a pyridine ring with a single bond, tends to change significantly in structure before and after the redox reaction. In contrast, the present organic compound forms a fused ring skeleton, and its x-conjugated portion is planar in the ground state. Thus, the structure of the present organic compound is not changed much by the redox reaction. In other words, the decomposition of the present organic compound by the redox reaction can be reduced because of the small structural change before and after the redox reaction. Hence, the present organic compound is excellent in durability.

Additionally, the present organic compound may have the following features:
(1-3) containing a substituent increasing the solubility in solvents.

The present organic compound may be dissolved in a solvent before use, as described later. Therefore, the present organic compound may have a substituent increasing the solubility in solvents.

In some embodiments, the present organic compound has a bulky substituent from the viewpoint of increasing its solubility in solvents. Examples of the bulky substituent include alkyl groups with 1 to 7 carbon atoms, alkoxy groups with 1 to 7 carbon atoms, aryl groups with 6 to 18 carbon atoms, and aryloxy groups with 6 to 10 carbon atoms. More specifically, the bulky substituent may be isopropyl, tert-butyl, methoxy, ethoxy, isopropoxy, tert-butoxy, terphenyl, or phenoxy.

Also, when used after being dissolved in a polar solvent, the present organic compound may have a substituent that increases the polarity. Examples of such a substituent include alkoxy groups with 1 to 7 carbon atoms and aryloxy groups with 6 to 10 carbon atoms. More specifically, such a substituent may be methoxy, ethoxy, isopropoxy, tert-butoxy, or phenoxy. These substituents increase the polarity of the present organic compound, increasing the solubility of the present organic compound in solvents.

The method for producing the present organic compound will now be described.

The present organic compound may be produced in any process without particular limitation and may be produced, for example, in the following process. For the organic compound in which at least one of X₁ and X₂ is an alkyl or aralkyl group, an organic compound represented by the following general formula (3) is reacted with a halide in a given solvent. Then, the resulting compound is subjected to an anion exchange reaction with a salt containing a desired anion in a given solvent to yield the desired organic compound.

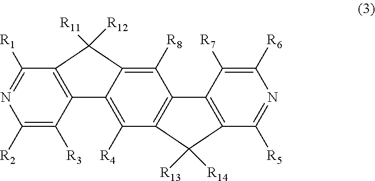

For the organic compound in which at least one of X₁ and X₂ is an aryl group, an organic compound represented by the above general formula (3) is allowed to react with a hypervalent iodine compound. Then, the resulting compound is subjected to an anion exchange reaction with a salt containing an anion in a given solvent to yield the desired organic compound. Only the imine on one side may be reacted by selecting the solvent and reaction temperature. Repeating reactions enable different substituents to be introduced to the two imine moieties.

The organic compound represented by general formula (3) may be produced in any process without particular limitation and may be produced, for example, in the following process. In the following synthesis route, R₁ to R₈ and R₁₁ to R₁₄ represent hydrogen atoms or substituents as in general formula (1).

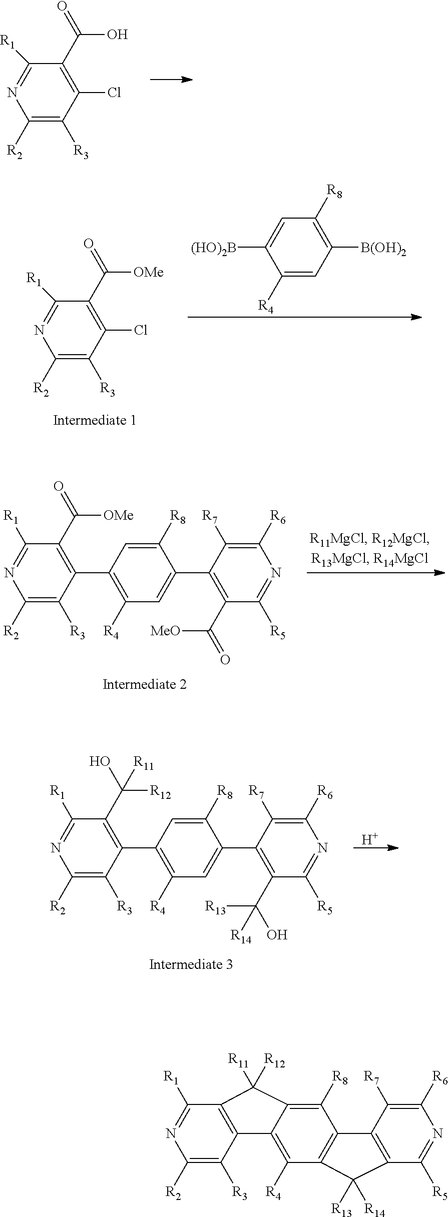

Intermediate 1 can be synthesized by methylating a 4-nicotinic acid derivative with a methylating agent. Intermediate 2 can be synthesized by coupling Intermediate 1 with a phenylenediboronic acid derivative. Asymmetric molecules may be synthesized by reacting different Intermediates 1, one after another, with a phenyldiboronic acid derivative. Intermediate 3 can be synthesized by reacting a Grignard reagent with Intermediate 2. The organic compound represented by general formula (3) can be synthesized by cyclizing Intermediate 3 in the presence of acid.

Specific structural formulas of the present organic compound are presented below, but the present organic compound is not limited to the following.

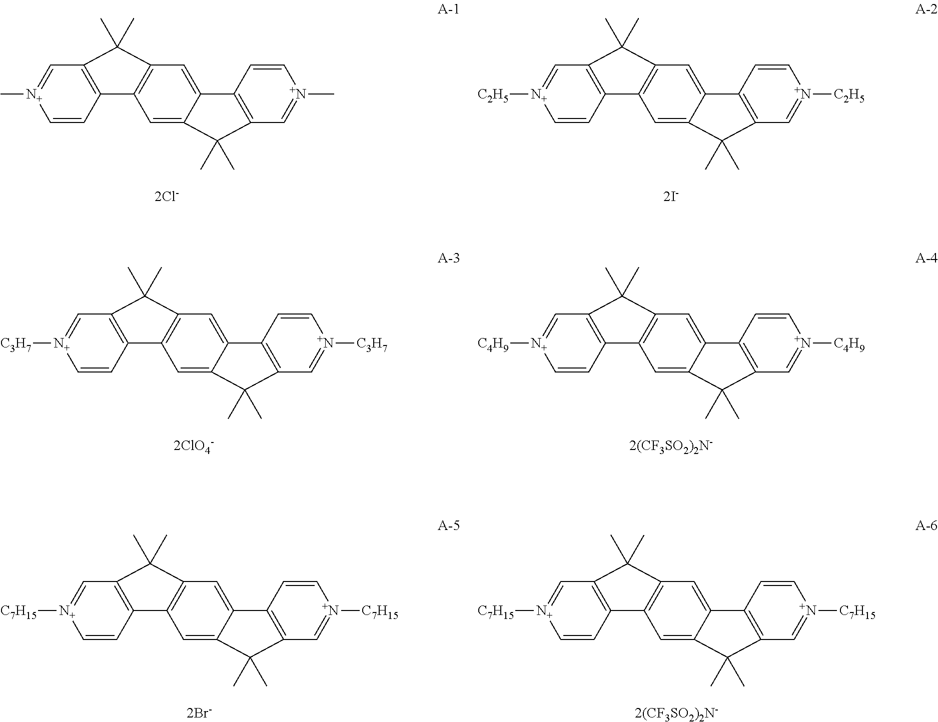

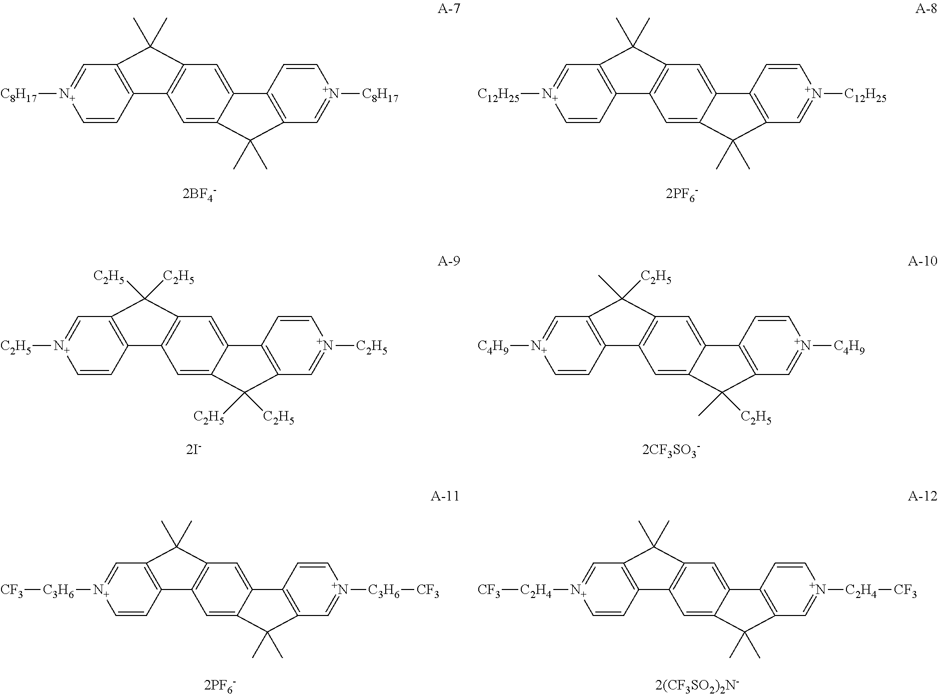

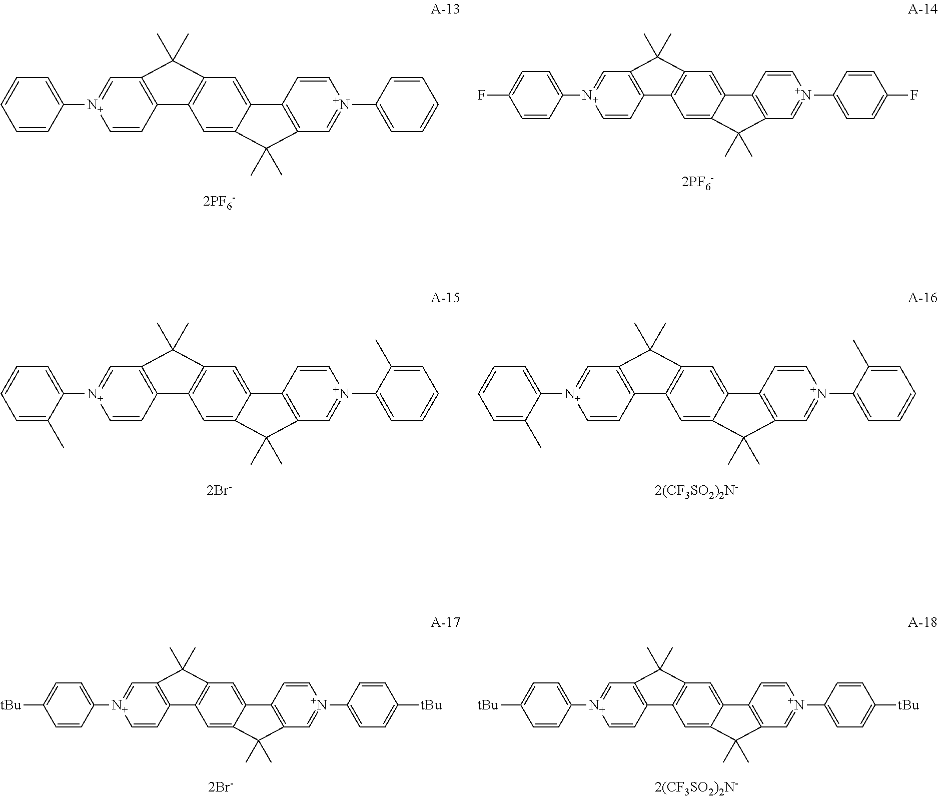

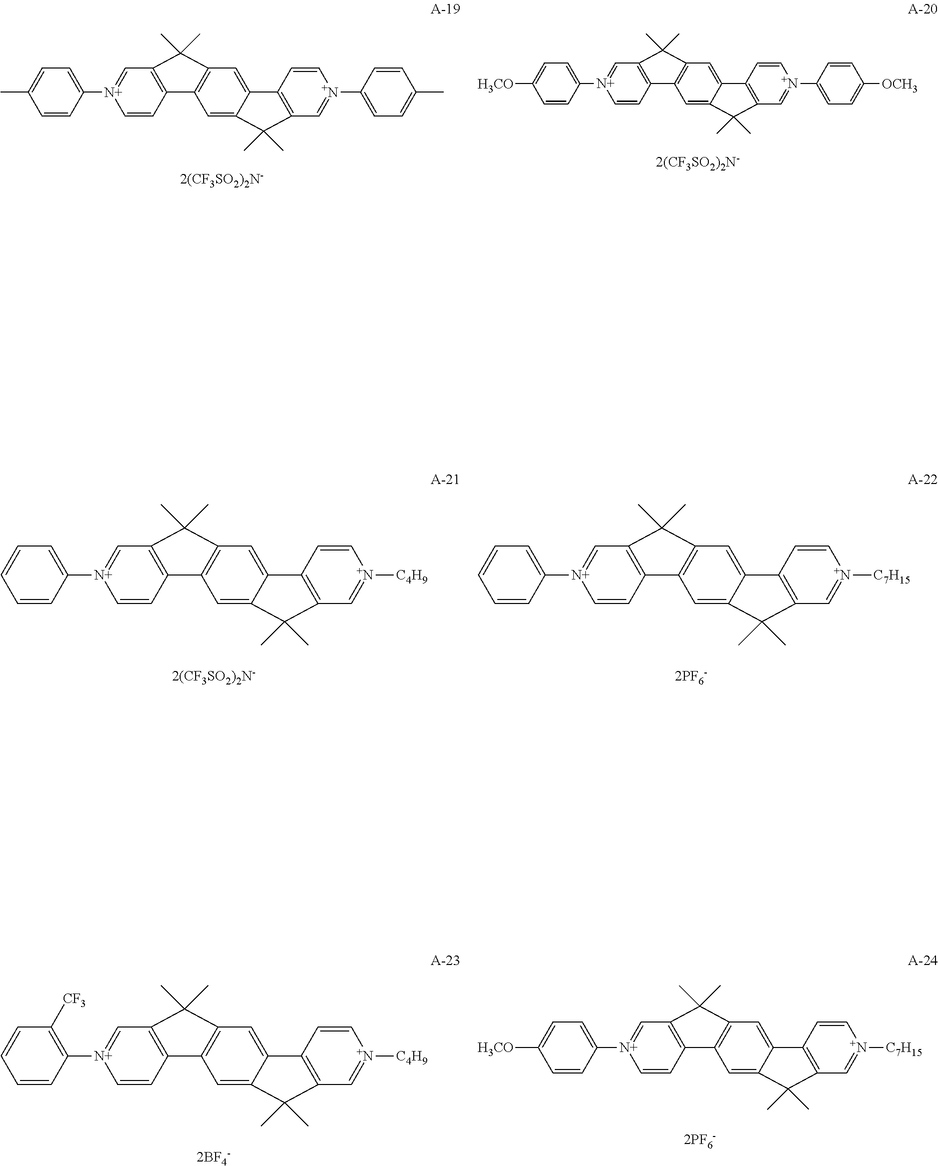

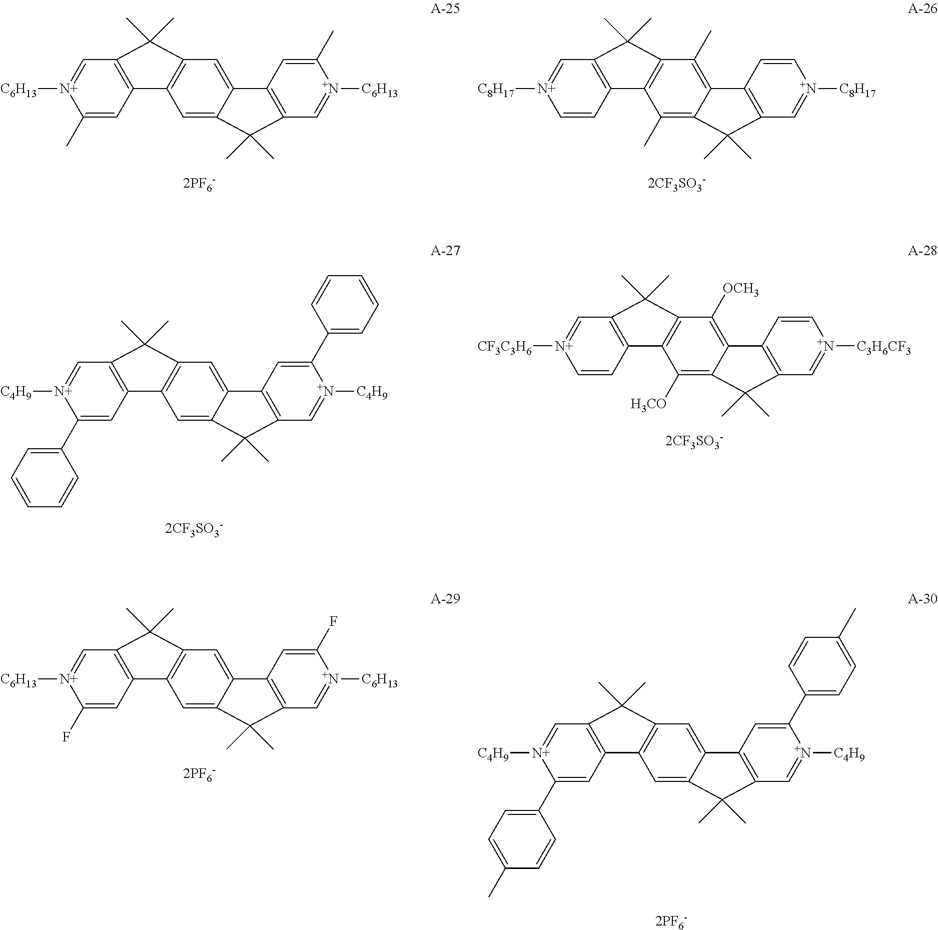

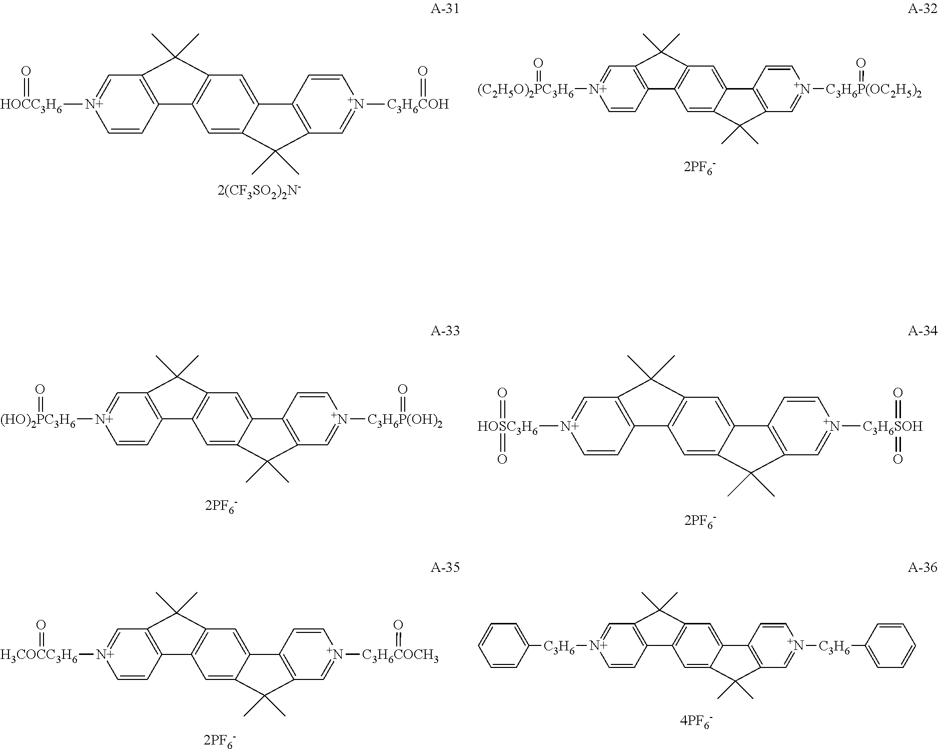

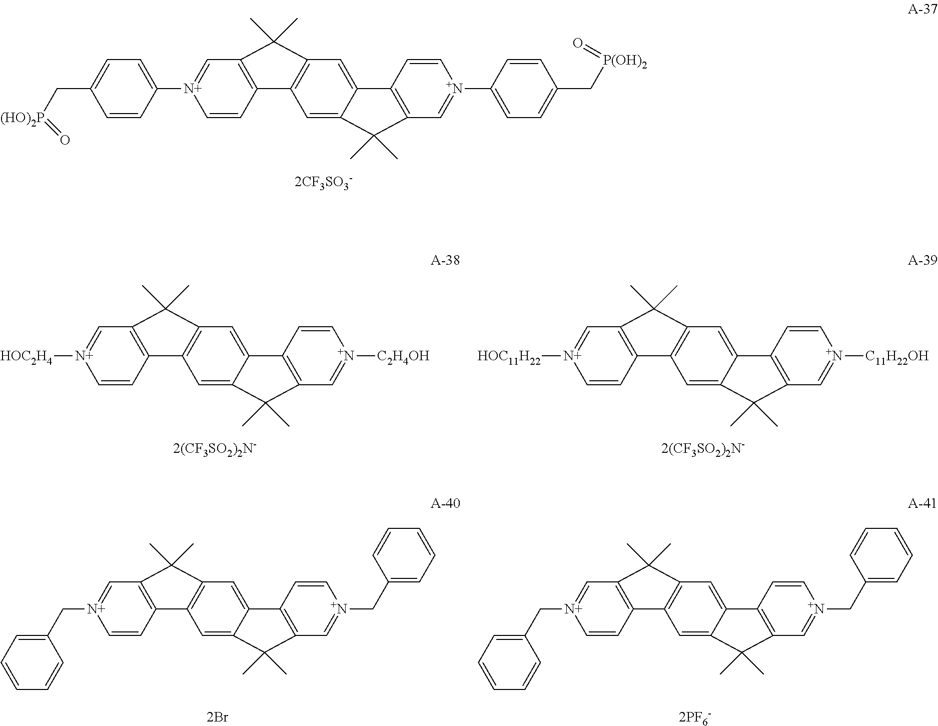

Among the example compounds above, the compounds in which X₁ and X₂ have the same structure are easy to synthesize. Specifically, A-1 to A-20 and A-25 to A-41 are such compounds.

Among the example compounds above, the compounds in which X₁ and X₂ have alkyl groups are excellent in durability. Specifically, A-1 to A-12, A-21 to A-35, A-38,and A-39 are such compounds.

Among the example compounds above, the compounds in which X₁ and X₂ have aryl groups can easily control the absorption wavelength. Specifically, A-13 to A-23 and A-21 to A-24 are such compounds.

The above example compounds, which are represented by general formula (1), are all organic compounds having stable structures in the first reduced state.

### EC Element

The present organic compound can be used in an EC layer of an EC element. An EC element according to an embodiment will now be described with reference to the drawings.

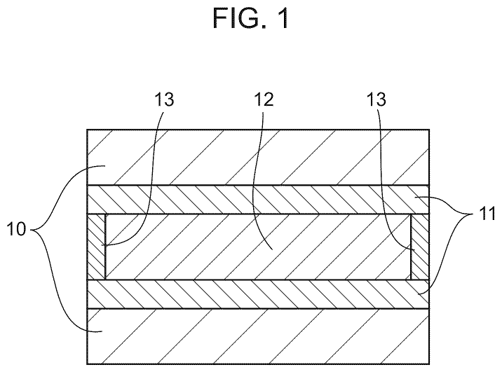

An EC element 1 depicted in FIG. 1 includes a pair of electrodes 11 and an EC layer 12 disposed between the pair of electrodes 11. The EC element 1 may further include a pair of transparent substrates 10. The EC element 1 may also include a spacer 13 that keeps the distance between the pair of electrodes 11 constant.

The EC layer 12 contains the present organic compound. The EC layer 12 may include a sub-layer made of the present organic compound and a sub-layer containing an electrolyte. Alternatively, the EC layer 12 may be provided as a solution containing an EC compound, namely the present organic compound, and an electrolyte. When the EC layer 12 is a solution layer, the entirety of the present organic compound, the solution, and other solutes may be referred to as an EC medium.

The components of the EC element 1 of the present embodiment will now be described.

### Substrates 10

The substrates 10 may be made of, for example, colorless or colored transparent resins, as well as colorless or colored glass or tempered glass. In some embodiments, the substrates 10 are transparent. Transparent substrates used herein refer to those exhibiting a visible light transmittance of 90% or more. Examples of the material of the substrates include polyethylene terephthalate, polyethylene naphthalate, polynorbornene, polyamide, polysulfone, polyether sulfone, polyether ether ketone, polyphenylene sulfide, polycarbonate, polyimide, and polymethyl methacrylate.

### Electrodes 11

Exemplary materials of the electrodes 11 include metals and metal oxides, such as indium tin oxide alloy (ITO), fluorine-doped tin oxide (FTO), tin oxide (NESA), indium zinc oxide (IZO), silver oxide, vanadium oxide, molybdenum oxide, gold, silver, platinum, copper, indium, and chromium; silicon-based materials, such as polycrystalline silicon and amorphous silicon; and carbon materials, such as carbon black, graphite, and glassy carbon. In some embodiments, the electrodes 11 are transparent. Transparent electrodes used herein refer to those exhibiting a visible light transmittance of 90% or more.

Conductive polymers whose electric conductivity is increased by doping or the like may also be used as well, and examples include polyaniline, polypyrrole, polythiophene, polyacetylene, poly (para-phenylene), and complexes of polyethylenedioxythiophene (PEDOT) and polystyrene sulfonic acid.

The electrodes 11 may be porous. In some embodiments, the porous electrodes are made of materials with a large surface area, such as microporous materials whose surfaces and interior have micropores, rod-shaped materials, or wire-like materials. For example, the porous electrodes are made of a metal, a metal oxide, or carbon. In some embodiments, the porous electrodes are made of a metal oxide, such as titanium oxide, tin oxide, iron oxide, strontium oxide, tungsten oxide, zinc oxide, tantalum oxide, vanadium oxide, indium oxide, nickel oxide, manganese oxide, or cobalt oxide.

### EC Layer 12

Examples of the electrolyte are as follows. When a liquid electrolyte is used, the electrolyte contains an ionically dissociable salt. Any compound exhibiting high solubility in the solvent can be used as the ionically dissociable salt without limitation. When a solid electrolyte is used, any compound compatible with the present organic compound can be used without limitation. In some embodiments, electron-donating electrolytes may be used. These electrolytes may be referred to as supporting electrolytes. For example, the electrolyte may be an inorganic ionic salt such as an alkali metal salt or an alkaline-earth metal salt, a quaternary ammonium salt, or a cyclic quaternary ammonium salt. More specifically, examples of the electrolyte include alkali metal (Li, Na, or K) salts, such as LiClO₄, LiSCN, LiBF₄, LiAsF₆, LiCF₃SO₃, LiPF₆, LiI, NaI, NaSCN, NaClO₄, NaBF₄, NaAsF₆, KSCN, and KCl; and quaternary ammonium salts and cyclic quaternary ammonium salts, such as (CH₃)₄NBF₄, (C₂H₅)₄NBF₄, (n-C₄H₉)₄NBF₄, (n-C₄H₉)₄NPF₆, (C₂H₅)₄NBr, (C₂H₅)₄NClO₄, and (n-C₄H₉)₄NClO₄.

The solvent used to dissolve the present organic compound and the electrolyte is not limited, provided that it can dissolve these compounds. Examples of the solvent include water and organic solvents, such as methanol, ethanol, propylene carbonate, ethylene carbonate, dimethyl sulfoxide, dimethoxyethane, γ-butyrolactone, γ-valerolactone, sulfolane, dimethylformamide, tetrahydrofuran, acetonitrile, propionitrile, benzonitrile, dimethylacetamide, methylpyrrolidinone, and dioxolane.

The EC layer 12 may further contain a polymer or a gelling agent to increase the viscosity or may be gelled.

Such polymers and gelling agents may be referred to as thickeners. Adding a thickener increases the viscosity of the EC solution. High-viscosity EC solutions, in which the movement of molecules is suppressed, hamper the present organic compound from forming associations, reducing the temperature dependence of the absorption spectrum of the organic compound. In some embodiments, therefore, the EC solution contains a thickener.

However, EC solutions with excessively high viscosity do not allow the molecules in the EC solution to move easily, reducing the reaction speed of the EC element. Thus, EC solutions with excessively high viscosity are not suitable.

Specifically, the viscosity of the EC solution may be 10 cP to 5000 cP and, in some embodiments, 50 cP to 1000 cP. The viscosity of the EC solution may be 150 cP or less, for example, 100 cP or less or 65 cP or less. Also, the viscosity of the EC solution may be 20 cP or more, for example, 50 cP or more.

The proportion of the thickener may be 20 wt % or less to 100 wt % of the EC solution. In some embodiments, it may be 1 wt % to 15 wt % or 5 wt % to 10 wt %.

Examples of the polymer include, but are not limited to, polyacrylonitrile, carboxymethyl cellulose, polyvinyl chloride, polyalkylene oxide, polyurethane, polyacrylate, polymethacrylate, polyamide, polyacrylamide, polyester, and Nafion (registered trademark). In some embodiments, polymethyl methacrylate, polyethylene oxide, or polypropylene oxide may be used.

In an embodiment, the EC element may contain the present organic compound and one or more other organic compounds (first organic compound) different from the present organic compound. The first organic compound may be a single compound or a plurality of compounds and may be a compound that exhibits a color upon oxidation, a compound that exhibits a color upon reduction, or a compound having both characteristics. Organic compounds having the skeleton represented by general formula (1) color in the reduced state. Accordingly, the first organic compound may color in the oxidized state.

The present organic compound can provide an EC element that can absorb desired colors in combination with the first organic compound. When colors, the first organic compound has an absorption wavelength in the range of 400 nm to 800 nm, for example, 420 nm to 700 nm. Having an absorption wavelength in a specific range means that the absorption spectrum has a peak in the specific range. Combining the present organic compound with two or more first organic compounds can produce an EC element that absorbs light over the entire visible region to color black.

In some embodiments, the EC element contains four or more EC compounds, including the present organic compound. This is because filters including such an EC element tend to evenly absorb light with varying wavelengths.

Examples of the first organic compound are cited below.

First organic compounds that color upon oxidation include oligothiophene-based compounds; phenazine-based compounds, such as 5,10-dihydro-5,10-dimethylphenazine and 5,10-dihydro-5,10-diisopropylphenazine; metallocene-based compounds, such as ferrocene, tetra-t-butylferrocene, and titanocene; phenylenediamine-based compounds, such as N,N′,N,N′-tetramethyl-p-phenylenediamine; and pyrazoline-based compounds, such as 1-phenyl-2-pyrazoline.

First organic compounds that color upon reduction include viologen-based compounds, such as N,N′-diheptylbipyridinium diperchlorate, N,N′-diheptylbipyridinium ditetrafluoroborate, N,N′-diheptylbipyridinium dihexafluorophosphate, N,N′-diethylbipyridinium diperchlorate, N,N′-diethylbipyridinium ditetrafluoroborate, N,N′-diethylbipyridinium dihexafluorophosphate, N,N′-dibenzylbipyridinium diperchlorate, N,N′-dibenzylbipyridinium ditetrafluoroborate, N,N′-dibenzylbipyridinium dihexafluorophosphate, N,N′-diphenylbipyridinium diperchlorate, N,N′-diphenylbipyridinium ditetrafluoroborate, and N,N′-diphenylbipyridinium dihexafluorophosphate; anthraquinone-based compounds, such as 2-ethylanthraquinone, 2-t-butylanthraquinone, and octamethylanthraquinone; ferrocenium salt-based compounds, such as ferrocenium tetrafluoroborate and ferrocenium hexafluorophosphate; and styryl-based compounds.

Phenazine-based compounds used herein have chemical structures containing a 5,10-dihydrophenazine skeleton, including compounds containing 5,10-dihydrophenazine with one or more substituents. For example, the hydrogen atoms at the 5- and 10- positions of 5,10-dihydrophenazine may be substituted by alkyl groups with 1 to 8 carbon atoms or aryl groups with 6 to 10 carbon atoms. More specifically, such substituents include alkyl groups, such as methyl, ethyl, propyl, isopropyl, n-butyl, and tert-butyl, and aryl groups, such as phenyl and naphthyl.

In phenazine-based compounds, the 5,10-dihydrophenazine skeleton may have one or more substituents. Examples of substituents on the 5,10-dihydrophenazine skeleton include alkyl groups having 1 to 20 carbon atoms, aryl groups having 6 to 12 carbon atoms, and aryloxy groups having 6 to 10 carbon atoms. More specifically, such substituents include alkyl groups, such as methyl, ethyl, propyl, n-butyl, and tert-butyl; aryl groups, such as phenyl and naphthyl; and aryloxy groups, such as phenoxy.

The substituent of 5,10-dihydrophenazine skeleton may further have a substituent, such as alkyl groups having 1 to 4 carbon atoms or alkoxy groups having 1 to 4 carbon atoms. Examples of such a substituent include alkyl groups, such as methyl, ethyl, propyl, n-butyl, and tert-butyl; and alkoxy groups, such as methoxy and isopropoxy.

The same applies to substituents of other first organic compounds, such as viologen-based compounds.

In some embodiments, the first organic compound is selected from phenazine-based compounds, metallocene-based compounds, phenylenediamine-based compounds, and pyrazoline-based compounds.

The compounds contained in the EC layer of the EC element disclosed herein can be identified by known extraction and analysis methods. For example, the compounds may be extracted by chromatography and analyzed by nuclear magnetic resonance (NMR). When the EC layer is solid, the compounds can be analyzed by time-of-flight secondary ion mass spectrometry (TOF-SIMS) or the like.

### Spacer 13

The spacer 13 is disposed between the pair of electrodes 11 to define a space that accommodates the EC layer 12. The spacer 13 may be made of polyimide, polytetrafluoroethylene, fluorocarbon rubber, or epoxy resin. The spacer 13 keeps the distance between the electrodes of the EC element 1.

In an embodiment, the EC element may have a liquid inlet defined by the pair of electrodes 11 and the spacer 13. After a composition containing the present organic compound is introduced through the liquid inlet, the liquid inlet is covered with a sealing member and further sealed with an adhesive to obtain the EC element 1. The sealing member also serves to isolate the composition from the adhesive so as not to come into contact with each other. The sealing member may have any shape without limitation but is, in some embodiments, tapered like wedges or the like.

The EC element disclosed herein may be produced by any method without limitation and, for example, by introducing a liquid containing EC compounds prepared in advance into the space between the pair of electrodes 11, using vacuum injection, atmospheric injection, meniscus, or the like.

### Applications of EC Element

When operating, the EC element disclosed herein can adjust the amount of light passing through the EC layer. The EC element can be used in optical filters, lens units, imaging devices, window components, and the like.

### Optical Filter

An optical filter according to an embodiment of the present disclosure includes an EC element 1 and an active element connected to the EC element 1. The optical filter may include peripheral devices. The active element may be connected to the EC element 1 directly or indirectly with another element therebetween. The active element drives the EC element 1 and adjusts the amount of light passing through the EC element. The active element may be, for example, a transistor. The transistor may contain an oxide semiconductor, such as InGaZnO, in the active region.

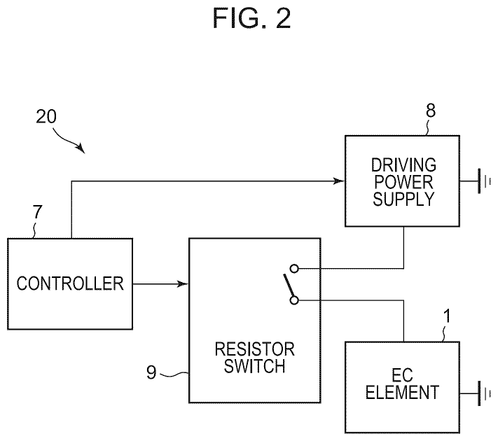

The optical filter in the present embodiment includes the EC element disclosed herein and a drive unit connected to the EC element. FIG. 2 is a schematic diagram of an example of a drive unit 20 and the EC element 1 driven by the drive unit 20. In the illustrated embodiment, the drive unit 20 further includes a controller 7, a driving power supply 8, and a resistor switch 9.

The driving power supply 8 applies a voltage required for the electrochemical reaction of the EC compounds in the EC layer 12 (hereinafter also referred to as “driving voltage”) to the EC element 1. In some embodiments, the driving voltage is constant. This is because when the EC layer 12 contains a plurality of EC compounds, the absorption spectrum may change due to the differences in redox potential and molar absorption coefficient among the EC compounds. Driving voltage application is started and kept by signals from the controller 7. In an embodiment, the application of a constant voltage is maintained while the optical transmittance of the EC element 1 is controlled.

The controller 7 controls the transmittance of the EC element 1 in a suitable way for the EC element 1. More specifically, conditions previously required for a desired transmittance may be input to the EC element 1, or conditions to meet the set value of the transmittance may be selected according to the comparison between the set transmittance and the transmittance of the EC element 1. Parameters to be varied include voltage, current, and duty cycle. The term duty cycle used herein refers to the proportion of one period of the applied voltage duration within the period of a pulse voltage waveform. The controller 7 can vary the voltage, current, or duty cycle to change the color density of the EC element 1.

In the embodiments disclosed herein, known techniques can be used to vary voltage and current and modulate the pulse width. Also, the pulse width may be modulated as described below.

The resistor switch 9 switches between a resistor R1 and a resistor R2 with higher resistance than R1 (both resistors not shown) and connects either resistor in series in a closed circuit including driving power supply 8 and EC element 1. The resistance of the resistor R1 may be lower than at least the highest impedance in the closed circuit of the element and is, in some embodiments, 10 Ω or less. The resistance of the resistor R2 may be higher than the highest impedance in the closed circuit of the element and is, in some embodiments, 1 MΩ or more. The resistor R2 may be air. In this instance, the closed circuit is open in strict meaning but can be considered closed, provided that air is interpreted as the resistor R2.

The controller 7 sends signals to the resistor switch 9 to control the switching between the resistors R1 and R2. When the resistor R1 is on, the EC element 1 colors, and when the resistor R2 is on, the EC element 1 is achromatized. While the resistor R2 is on, EC compounds self-achromatize. This self-achromatization is caused, for example, by instability of radicals of EC compounds produced in coloring, diffusion of the radicals to the opposing electrode with a different potential, or collision between anodic radicals and cathodic radicals in the solution.

### Lens Unit

The lens unit according to an embodiment of the disclosure includes an imaging optical system, and an optical filter including the EC element disclosed herein. The imaging optical system may include one or more lenses. The optical filter may be disposed between or among the lenses or outside the lenses. In some embodiments, the optical filter is disposed on the optical axis of the lenses.

### Imaging Device

The imaging device in an embodiment includes an optical filter including the EC element disclosed herein, and a light receiving element configured to receive light passing through the optical filter. Specific examples of the imaging device include cameras, video cameras, and camera phones. The imaging device may be such that a lens unit including one or more lenses is separable from the main body including the light-receiving element. In an embodiment in which the lens unit is separable from the main body of the imaging device, an optical filter, apart from the imaging device, may be used for imaging. At this time, the optical filter may be placed outside the lens unit, between the lens unit and the light-receiving element, or between or among the lenses (when the lens unit includes a plurality of lenses).

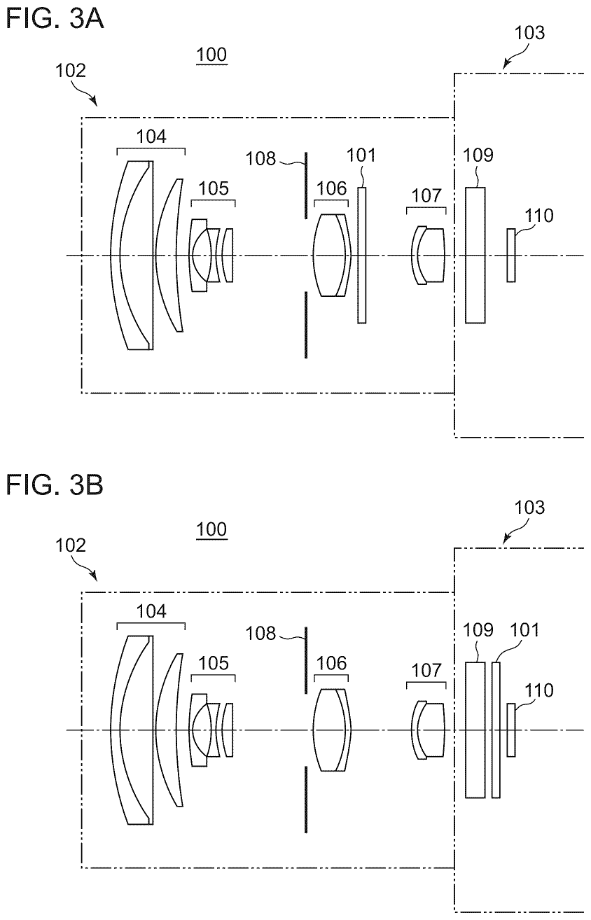

FIG. 3A is a schematic diagram of an example of an imaging device in which an optical filter is disposed in a lens unit, and FIG. 3B is a schematic diagram of an example of an imaging device in which an optical filter is disposed in an imaging unit.

The imaging device 100 includes a lens unit 102 and an imaging unit 103. The lens unit 102 includes an optical filter 101, and an imaging optical system including lenses. The optical filter 101 is the optical filter disclosed herein described above. The number of lenses may be single or plural.

For example, the lens unit 102 may act as a rear focusing zoom lens that focuses behind the diaphragm. The lens unit 102 includes four lens sets arranged in the following order from the imaging subject (object): a first lens set 104 having a positive refractive power, a second lens set 105 having a negative refractive power, a third lens set 106 having a positive refractive power, and a fourth lens set 107 having a positive refractive power. The imaging device may focus, for example, by varying the distance between the second lens set 105 and the third lens set 106 to vary magnification and moving some lenses of the fourth lens set 107.

For example, the lens unit 102 may include an aperture diaphragm 108 between the second lens set 105 and the third lens set 106 and the optical filter 101 between the third lens set 106 and the fourth lens set 107. The lens unit 102 is configured to allow light to pass through the lens sets 104 to 107, the diaphragm 108, and the optical filter 101 to adjust the amount of light with the aperture diaphragm 108 and the optical filter 101.

The lens unit 102 may be removably connected to the imaging unit 103 with a mounting member (not shown).

Although in the present embodiment, the optical filter 101 is disposed between the third lens set 106 and the fourth lens set 107 in the lens unit 102, the imaging device 100 is not limited by this configuration. For example, the optical filter 101 may be disposed in front of the aperture diaphragm 108 (on the imaging subject side), behind the aperture diaphragm (on the imaging unit 103 side), in front of or behind any of the first to fourth lens sets 104 to 107, or between any two of the lens sets. Placing the optical filter 101 at the position where light converges enables the decrease in the area of the optical filter 101.

The configuration of the lens unit 102 may also be selected as desired without being limited to that described above. The lens unit may be of a type other than the rear focusing type and may be, for example, of an inner focusing type that focuses on a position in front of the diaphragm. Also, the lens unit may act as a special lens as needed, such as a fisheye lens or a microlens, as well as the zoom lens.

The imaging unit 103 includes a glass block 109 and a light-receiving element 110. The glass block 109 may be a low-pass filter, a face plate, or a color filter. The light-receiving element 110 is a sensor that receives light passing through the lens unit, and an imaging element, such as CCD or CMOS, may be used. Alternatively, the light-receiving element may be a light sensor such as a photodiode or any other element or device capable of obtaining and outputting information such as light intensity or wavelength.

When the optical filter 101 is incorporated in the lens unit 102, as depicted in FIG. 3A, the drive unit 20 may be placed within or outside the lens unit 102. In the case of placing the lens unit 102 outside, the drive unit 20 is connected for drive control to the EC element 1 of the lens unit 102 with a wire.

In the above-described imaging device 100, the optical filter 101 is located inside the lens unit 102. However, the configuration is not limited to that in this embodiment, provided that the optical filter 101 is located at a position inside the imaging device 100 so that the light-receiving element 110 can receive the light passing through the optical filter 101.

For example, the imaging unit 103 may have the optical filter 101, as depicted in FIG. 3B. FIG. 3B is a schematic diagram of another example of the imaging device disclosed herein, illustrating a configuration in which the optical filter is located in the imaging unit 103. In FIG. 3B, the optical filter 101 is located, for example, right in front of the light-receiving element 110. In a configuration in which the imaging device itself incorporates an optical filter 101, the lens unit 102 to be connected need not include the optical filter 101. Accordingly, a dimmable imaging device can be configured using an existing lens unit 102.

The imaging device 100 disclosed herein can be applied to products including a combination of a light-receiving element and the function of adjusting the amount of light. For example, the imaging device may be a camera, a digital camera, a video camera, or a digital video camera and can also be used in products with built-in imaging devices, such as mobile phones, smartphones, PCs, and tablet computers.

In the imaging device 100 disclosed herein, an optical filter 101 can be used as a dimmer member to enable a single filter to vary the dimming level as appropriate. This is advantageous for reducing the number of components and saving space.

### Window

A widow according to an embodiment of the disclosure includes a pair of substrates, the EC element disclosed herein disposed between the pair of substrates, and an active element connected to the EC element. The window may adjust the amount of light passing through the EC element 1, in which the active element is connected as a driver to the EC element 1 for driving the EC element 1, but is not limited to this configuration. The active element may be a transistor. The transistor may contain a semiconductor material, such as InGaZnO, in the active region. The window disclosed herein may be called a variable transmittance window.

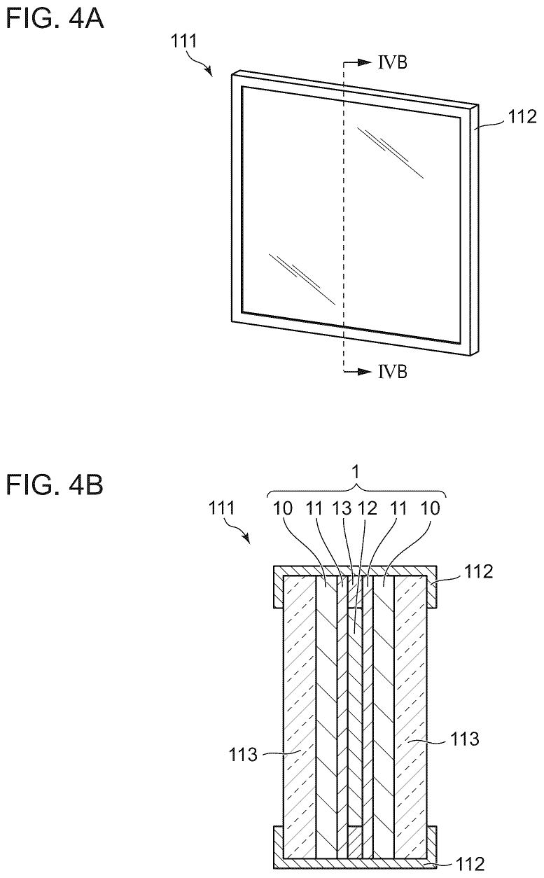

FIG. 4A is a schematic diagram of an example of a dimming window 111 used as a window using the EC element disclosed herein, and FIG. 4B is a schematic sectional view taken along line IVB-IVB in FIG. 4A. The dimming window disclosed herein includes an EC element 1 (optical filter) and transparent plates 113 holding the EC element in between, and a frame 112. The EC element 1 is provided with a drive unit (not shown). The drive unit may be integrated with the frame 112 or located outside the frame 112 and connected to the EC element 1 with a wire.

The frame 112 may be made of any material without limitation, and the entirety of a structure that covers at least a portion of the EC element 1 and has an integrated form may be considered the frame. While the EC element 1 depicted in FIG. 4B is a component member apart from the transparent plates 113, the substrates 10 of the EC element 1 may be considered transparent plates 113, for example.

The material of the transparent plates 113 is not limited, provided that the material has high light transmittance. In some embodiments, glass is used in view of use as a window.

The dimming window disclosed herein may be used, for example, to adjust the amount of sunlight entering a room during the daytime. The dimming window may also be used to adjust the amount of heat as well as the amount of sunlight, controlling the brightness and temperature in a room. Additionally, the dimming window may be used as a shutter to block views from the outside to the interior. In addition to glass windows for buildings, such a dimming window may be used as a window for vehicles such as cars, trains, airplanes, and ships.

The EC element may be provided with a reflection member in one of the light paths. Such a window is called an EC mirror. The EC mirror includes a pair of substrates, the EC element disclosed herein between the pair of substrates, an active element connected to the EC element, and the reflection member. The EC mirror may be provided as an anti-glare mirror in a car or the like.

Thus, the EC element 1 including the EC layer 12 containing the organic compound having the skeleton represented by general formula (1) or the organic compound represented by general formula (2) can be used in optical filters, lens units, imaging devices, and windows. In the optical filter, lens unit, imaging device, and window according to the present disclosure, the organic compound represented by general formula (1) or (2) may be combined with other EC compounds absorbing colors in other wavelength ranges. Thus, various colors can be absorbed.

Also, the disclosed optical filter, lens unit, imaging device, and window each contain the organic compound represented by general formula (1) or (2), thereby exhibiting increased transparency in the achromatizing state.

### EXAMPLES

The disclosure will be further described in detail with reference to the Examples below. However, the invention is not limited to the Examples disclosed below.

### Example 1

### Synthesis of Example Compound A-5

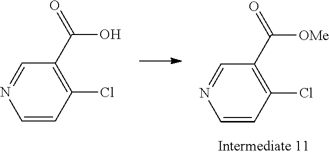 [SMILES: O=C(O)c1cnccc1Cl ; COC(=O)c1cnccc1Cl]

A reaction vessel was charged with 4-chloronicotinic acid (2.5 g, 15.7mmol), dichloromethane (50 mL) and cooled to 0° C. Oxalyl chloride (2.8 mL, 34.5 mmol) and N,N-dimethylformamide (2 mL) were added dropwise into the resulting solution, followed by increasing the temperature to room temperature and stirring the solution for 3 hours. This reaction solution was cooled to 0° C., and methanol (5 mL) was added dropwise. After concentrating the reaction solution, ethyl acetate was added to prepare a suspension. After filtration, the residue was washed with ethyl acetate to yield Intermediate 11 (2.7 g, 99% yield).

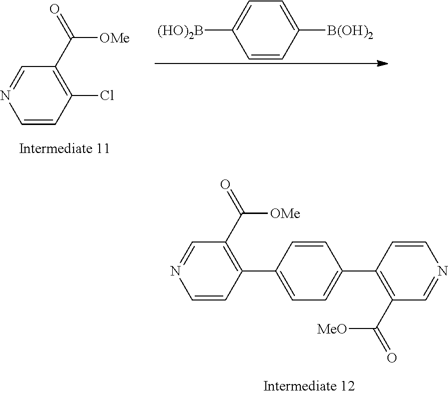 [SMILES: COC(=O)c1cnccc1Cl ; OB(O)c1ccc(B(O)O)cc1 ; COC(=O)c1cnccc1-c1ccc(-c2ccncc2C(=O)OC)cc1]

A reaction vessel was charged with Intermediate 11 (2.7 g, 15.7 mmol), 1,4-phenylenediboronic acid (1.3 g, 7.1 mmol), 1,4-dioxane (30 mL), and water (10 mL), and dissolved oxygen was removed with nitrogen. Then, tetrakis(triphenylphosphine)palladium(0) (0.87 g, 0.75 mmol) and tripotassium phosphate (8.4 g, 39.6 mmol) were added in a nitrogen atmosphere, followed by a reaction at 100° C. for 8 hours. After cooling the reaction solution, water and ethyl acetate were added, and the oil layer was extracted and concentrated. The resulting oil layer was then subjected to recrystallization with a toluene/ethyl acetate mixed solvent to yield Intermediate 12 (1.8 g, 70% yield).

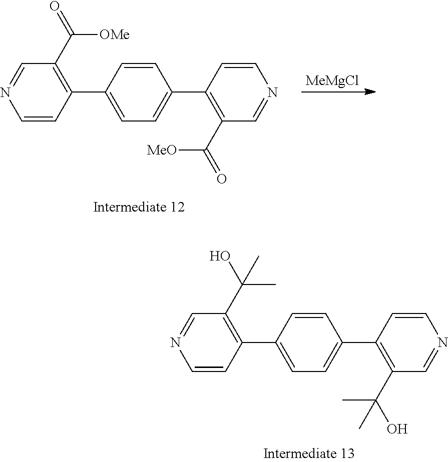 [SMILES: "MeMgCl" ; COC(=O)c1cnccc1-c1ccc(-c2ccncc2C(=O)OC)cc1 ; CC(C)(O)c1cnccc1-c1ccc(-c2ccncc2C(C)(C)O)cc1]

A reaction vessel was charged with Intermediate 12 (1.6 g, 4.6 mmol) and anhydrous tetrahydrofuran (60 mL) and cooled to −5° C. Then, 36.8 mL of methylmagnesium bromide solution (about 1.0 M in tetrahydrofuran, 36.8 mmol) was slowly added dropwise. After being stirred for 1 hour, the solution was warmed to room temperature and stirred for 6 hours. After stopping the reaction by adding an ammonium chloride aqueous solution, diethyl ether was added. The thus-formed oil layer was extracted and concentrated. The resulting oil layer was then subjected to recrystallization with a toluene/ethanol mixed solvent to yield Intermediate 13 (1.2 g, 75% yield).

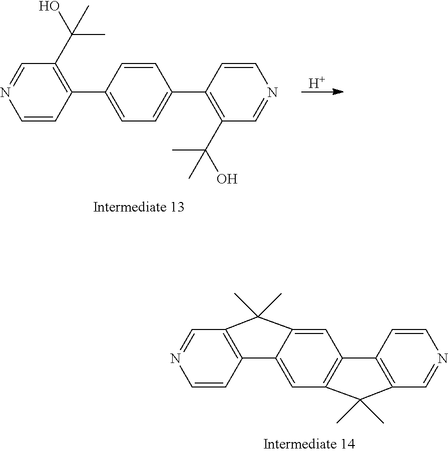

A reaction vessel was charged with Intermediate 13 (600 mg, 1.7 mmol) and trifluoromethanesulfonic acid (5 mL), followed by stirring at room temperature for 5 hours. The resulting solution was neutralized with a sodium hydrogencarbonate aqueous solution, and the precipitated crystals were collected and subjected to recrystallization with 2-propanol/hexane mixed solvent to yield Intermediate 14 (484 mg, 90% yield).

The structure of Intermediate 14 was identified by ¹H NMR analysis. ¹H NMR (CDCl₃, 500 MHz) δ (ppm): 8.77 (s, 2H), 8.66 (d, 2H), 7.89 (s, 2H), 7.70 (d, 2H), 1.69 (s, 12H).

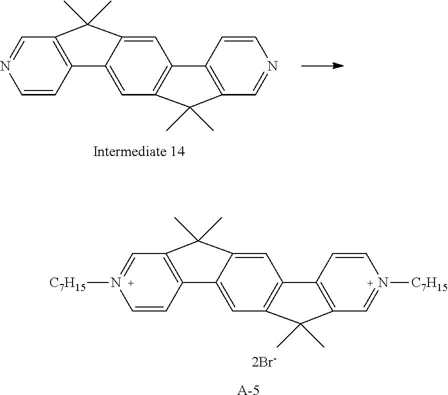

A reaction vessel was charged with Intermediate 14 (174 mg, 0.5 mmol), 1-bromoheptane (448 mg, 2.5 mmol), and N,N-dimethylformamide (5 mL), followed by a reaction at 100° C. for 24 hours. After completion of the reaction, precipitated solids were collected by filtration and washed with ethyl acetate to yield 0.30 g of Example Compound A-5 (88% yield).

The structure of Example Compound A-5 was identified by ¹H NMR analysis.

¹H NMR (DMSO-d6, 500 MHz) δ (ppm): 9.52 (s, 2H), 9.16 (d, 2H), 8.79 (s, 2H), 8.70 (d, 2H), 4.62 (t, 4H), 2.00 (m, 4H), 1.68 (s, 12H), 1.39-1.21 (m, 16H), 0.87 (t, 6H)

### Example 2

### Synthesis of Example Compound A-6

Example Compound A-5 (201 mg, 0.3mmol) was dissolved in water. An aqueous solution in which lithium bis (trifluoromethanesulfonyl) imide (0.43 g, 1.5 mmol) was dissolved was added dropwise, followed by stirring at room temperature for 3 hours. Precipitated crystals were collected by filtration and washed with isopropyl alcohol and diethyl ether in this order to yield 283 mg of Example Compound A-6 (88% yield).

The structure of Example Compound A-4 was identified by ¹H NMR analysis.

¹H NMR (CD₃CN, 500 MHz) δ (ppm): 8.85 (s, 2H), 8.66 (d, 2H), 8.47 (s, 2H), 8.37 (d, 2H), 4.54 (t, 4H), 2.04 (m, 4H), 1.71 (s, 12H), 1.44-1.28 (m, 16H), 0.92 (t, 6H)

### Example 3

### Synthesis of Example Compound A-40

A reaction vessel was charged with Intermediate 14 (174 mg, 0.5 mmol), benzyl bromide (342 mg, 2.0 mmol), and acetonitrile (10 mL), followed by a reaction at 80° C. for 8 hours. After completion of the reaction, precipitated solids were collected by filtration to yield 0.30 g of Example Compound A-40 (92% yield).

The structure of Example Compound A-40 was identified by ¹H NMR analysis.

¹H NMR (D20, 500 MHz) δ (ppm): 10.04 (s, 2H), 8.79 (d, 2H), 8.38 (s, 2H), 8.34 (d, 2H), 7.45 (s, 10H), 5.75 (s, 4H), 1.59 (s, 12H)

### Example 4

### Synthesis of Example Compound A-41

Example Compound A-40 (0.13 g, 0.2 mmol) was dissolved in water, and an aqueous solution in which ammonium hexafluorophosphate (200 mg) was dissolved was added dropwise, followed by stirring at room temperature for 3 hours. Precipitated crystals were collected by filtration and washed with water, isopropyl alcohol, and diethyl ether in this order to yield Example Compound A-41 (140 mg, 89% yield).

The structure of Example Compound A-41 was identified by ¹H NMR analysis.

¹H NMR (CD₃CN, 500 MHz) δ (ppm): 8.98 (s, 2H), 8.74 (d, 2H), 8.47 (s, 2H), 8.39 (d, 2H), 7.52 (s, 10H), 5.75 (s, 4H), 1.70 (s, 12H)

### Example 5

### Synthesis of Example Compound A-17

A reaction vessel was charged with Intermediate 14 (174 mg, 0.5 mmol), di-tert-butylphenyliodonium hexafluorophosphate (2.69 g, 5 mmol), copper (II) acetate monohydrate (18 mg, 0.10 mmol), and N,N-dimethylformamide (2.5 mL), followed by a reaction at 100° C. for 48 hours. After completion of the reaction, the reaction solution was concentrated under reduced pressure, and a solution (10 mL) of tetrabutylammonium bromide (2 g) in acetonitrile was added. Precipitated solids were collected by filtration to yield Example Compound A-17 (0.24 g, 65% yield).

The structure of Example Compound A-17 was identified by ¹H NMR analysis.

¹H NMR (D20, 500 MHz) δ (ppm): 9.21 (s, 2H), 8.93 (d, 2H), 8.52 (d, 2H), 8.51 (s, 2H), 7.72 (d, 4H), 7.58 (d, 4H), 5.75 (s, 4H), 1.66 (s, 12H), 1.40 (s, 18H)

### Example 6

### Synthesis of Example Compound A-18

Example Compound A-17 (0.15 g, 0.2 mmol) was dissolved in water, and an aqueous solution in which 0.3 g of lithium bis (trifluoromethanesulfonyl) imide was dissolved was added dropwise, followed by stirring at room temperature for 3 hours. Precipitated crystals were collected by filtration and washed with water, isopropyl alcohol, and diethyl ether in this order to yield Example Compound A-18 (212 mg, 93% yield).

The structure of Example Compound A-18 was identified by ¹H NMR analysis.

¹H NMR (CD₃CN, 500 MHz) δ (ppm): 9.12 (s, 2H), 8.93 (d, 2H), 8.59 (s, 2H), 8.55 (d, 2H), 7.83 (d, 4H), 7.75 (d, 4H), 5.75 (s, 4H), 1.80 (s, 12H), 1.45 (s, 18H)

### Example 7

Fabrication and Property Evaluation of EC Element

An electrolyte, tetrabutylammonium perchlorate, was dissolved to 0.1 M in propylene carbonate, and Example Compound A-6 in Example 2 was subsequently dissolved to 40.0 mM to prepare an EC solution.

Then, a pair of glass substrates with a transparent conductive film (ITO) were each provided with an insulating layer (SiO₂) on the four ends. A PET film (Melinex (registered trademark) S manufactured by Dupont Teijin Films, 125 μm thick) was disposed between the pair of transparent conductive film-provided glass substrates to define a gap between the substrates. Then, the substrates and the PET film were bonded with an epoxy adhesive for sealing, leaving an inlet through which the EC medium would be introduced. Thus, a hollow cell with an inlet was fabricated.

Then, the EC solution prepared above was introduced into the cell through the inlet by vacuum injection, and the inlet was sealed with the epoxy adhesive to obtain an EC element.

The EC element immediately after fabrication exhibited a transmittance of about 80% over the entire visible region and was thus highly transparent.

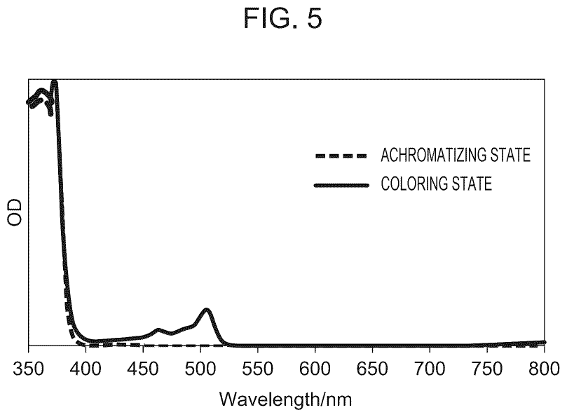

When a voltage of 2.5 V was applied, the EC element exhibited an absorption (λmax=508 nm) derived from a reduced form of Example Compound A-6 and colored. When −0.5 V was further applied, the EC element achromatized. This element can change reversibly between a coloring state and an achromatizing state. FIG. 5 illustrates UV-visible absorption spectra of the element fabricated in Example 3. The light source was a DH-2000S light source using deuterium and halogen lamps, manufactured by Ocean Optics, Inc.

### Example 8

An element was fabricated in the same manner as Example 7, except for using Example Compound A-41 instead of Example Compound A-6, which was used in Example 7. When a voltage of 3.0 V was applied, the element exhibited an absorption (λmax=511 nm) derived from a reduced form of Example Compound A-41.

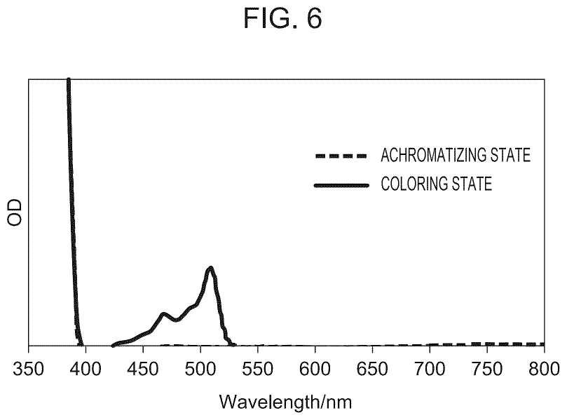

When −0.5 V was further applied, the EC element achromatized. This element can change reversibly between a coloring state and an achromatizing state. FIG. 6 illustrates UV-visible absorption spectra of the element fabricated in Example 8.

### Example 9

An element was fabricated in the same manner as Example 7, except for using Example Compound A-18 instead of Example Compound A-6, which was used in Example 7. When a voltage of 2.8 V was applied, the element exhibited an absorption (λmax=536 nm) derived from a reduced form of Example Compound A-18.

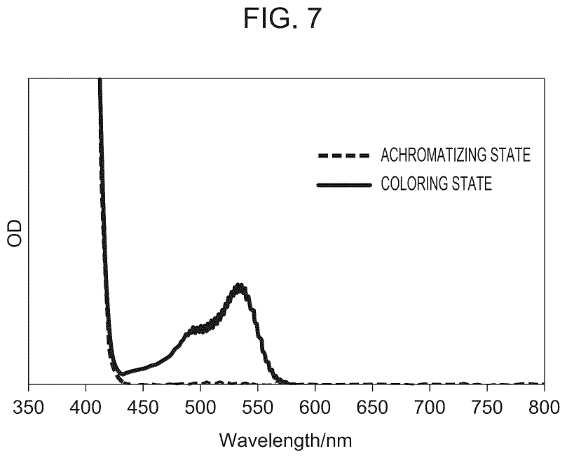

When −0.5 V was further applied, the EC element achromatized. This element can change reversibly between a coloring state and an achromatizing state. FIG. 7 illustrates UV-visible absorption spectra of the element fabricated in Example 9.

### Examples 10 to 12, Comparative Examples 1 and 2

### Reduction Potential Measurements of EC Elements

An electrolyte, tetrabutylammonium perchlorate, was dissolved to 0.1 M in propylene carbonate, and Example Compound A-13 in Example 2 was subsequently dissolved to 0.5 mM to prepare an EC solution. Similarly, Example Compound A-41 in Example 11, Example Compound A-18 in Example 12, Compound 1-a in Comparative Example 1, and Compound 1-b in Comparative Example 2 were dissolved to 0.5 mM to prepare EC solutions.

The reduction potential of the EC solutions was measured with an electrochemical analyzer Model 832B, manufactured by BAS Inc. The measurement used carbon as the working electrode, platinum as the counter electrode, and Ag/Ag⁺ electrode (solution of silver hexafluorophosphate in propylene carbonate) as the reference electrode. Ferrocene was also used as the internal standard. The results are presented in the following Table.

TABLE

| | | First | Second | |
|---|---|---|---|---|
| | | reduction | reduction | |
| | | potential | potential | Difference |
| | Structural formula | (A) | (B) | (A-B) |
| Example 10 | 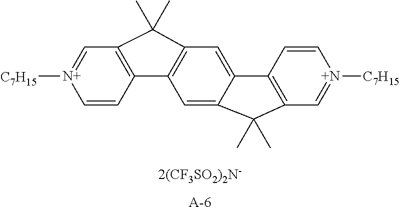 | −1.30 V | −1.52 V | 0.22 V |
| Example 11 | 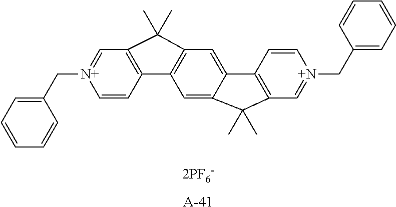 [SMILES: CC1(C)c2cc3c(cc2-c2cc[n+](Cc4ccccc4)cc21)C(C)(C)c1c[n+](Cc2ccccc2)ccc1-3 ; "A-41"] | −1.24 V | −1.46 V | 0.22 V |
| Example 12 | 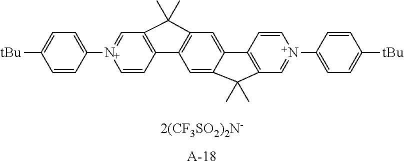 | −1.20 V | −1.35 V | 0.15 V |
| Comparative Example 1 | 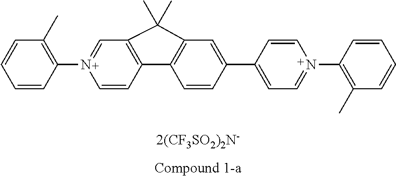 | −1.15 V | −1.27 V | 0.12 V |
| Comparative Example 2 | 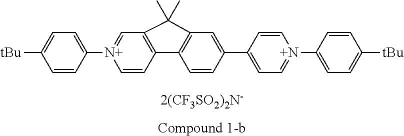 [SMILES: CC(C)(C)c1ccc(-[n+]2ccc(-c3ccc4c(c3)C(C)(C)c3c[n+](-c5ccc(C(C)(C)C)cc5)ccc3-4)cc2)cc1 ; "Compound 1-b"] | −1.11 V | −1.21 V | 0.10 V |

In the Table, Compound 1-a, which is an organic compound disclosed in PTL 1, exhibited a difference of 0.12 V between the first reduction potential and the second reduction potential, and Compound 1-b in Comparative Example 2 exhibited a difference of 0.10 V between the first reduction potential and the second reduction potential. In contrast, Example Compounds A-6, A-41, and A-18, which are organic compounds according to the present disclosure, exhibited differences of 0.22 V, 0.22 V, and 0.15 V, respectively, between the first reduction potential and the second reduction potential. Thus, the organic compounds disclosed herein have more stable structures in the first reduced state. More specifically, the organic compounds disclosed herein have larger differences between the first and second reduction potentials.

Hence, the organic compound of the present disclosure has a more stable structure in the first reduced state. Also, the organic compound of the present disclosure can absorb light with wavelengths of around 500 nm. Furthermore, the organic compound of the present disclosure is excellent in durability.

The present disclosure provides an organic compound that is more stable in terms of absorbable light wavelength.

While the present disclosure has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2023-209234 filed Dec. 12, 2023 and No. 2024-090294 filed Jun. 3, 2024, which are hereby incorporated by reference herein in their entirety.

What is claimed is:

1. An organic compound represented by formula (1): 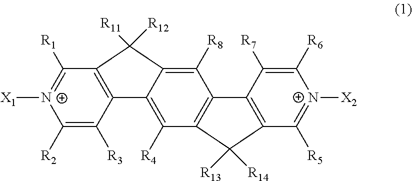
wherein in formula (1), X₁ and X₂ are each independently selected from the group consisting of substituted or unsubstituted alkyl groups, substituted or unsubstituted aryl groups, and substituted or unsubstituted aralkyl groups, R₁ to R₈ are each independently selected from the group consisting of a hydrogen atom, a deuterium atom, halogen atoms, substituted or unsubstituted alkyl groups, substituted or unsubstituted alkoxy groups, and substituted or unsubstituted aryl groups, and R₁₁ to R₁₄ are each independently selected from the group consisting of a hydrogen atom, substituted or unsubstituted alkyl groups, and substituted or unsubstituted aryl groups.

2. The organic compound according to claim 1, containing monovalent anions A₁⁻ and A₂⁻ as counter ions.

3. The organic compound according to claim 2, wherein A₁⁻ and A₂⁻ are each independently selected from the group consisting of Cl⁻, Br⁻, I⁻, BF₄⁻, PF₆⁻, ClO₄⁻, CF₃SO₃⁻, and (CF₃SO₂)₂N⁻.

4. The organic compound according to claim 3, wherein A₁⁻ and A₂⁻ are the same anion.

5. The organic compound according to claim 1, wherein in formula (1), X₁ and X₂ are each independently selected from the group consisting of alkyl groups with 1 to 12 carbon atoms, aryl groups with 6 to 10 carbon atoms, and aralkyl groups with 7 to 10 carbon atoms.

6. The organic compound according to claim 1, wherein in formula (1), X₁ and X₂ are each independently selected from the group consisting of alkyl groups with 1 to 10 carbon atoms and aryl groups with 6 to 10 carbon atoms.

7. The organic compound according to claim 1, wherein in formula (1), X₁ and X₂ have the same structure.

8. The organic compound according to claim 1, wherein in formula (1), R₁ to R₈ are each independently selected from the group consisting of a hydrogen atom, halogen atoms, alkyl groups with 1 to 4 carbon atoms, aryl groups with 6 to 10 carbon atoms, and alkoxy groups with 1 to 4 carbon atoms.

9. The organic compound according to claim 1, wherein in formula (1), R₁ to R₈ are each independently selected from the group consisting of a hydrogen atom, a fluorine atom, and methyl, phenyl, and methoxy groups.

10. The organic compound according to claim 1, wherein in formula (1), R₁₁ to R₁₄ are each independently selected from the group consisting of a hydrogen atom, alkyl groups with 1 to 4 carbon atoms, and aryl groups with 6 to 10 carbon atoms.

11. The organic compound according to claim 1, wherein in formula (1), R₁₁ to R₁₄ are each independently selected from the group consisting of a hydrogen atom, alkyl groups with 1 to 4 carbon atoms, and a phenyl group.

12. An organic compound that exhibits a color when reduced and exhibits a plurality of reduction peaks including a first reduction potential that is the largest reduction potential and a second reduction potential that is the second largest reduction potential in cyclic voltammetry measurement,
wherein the difference between the first reduction potential and the second reduction potential is larger than 0.12 V.

13. The organic compound according to claim 12, wherein the organic compound is represented by the following formula (2): 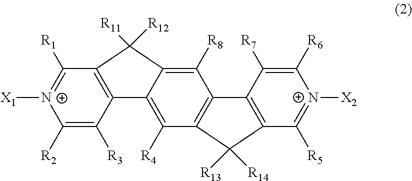
wherein in formula (2), X₁ and X₂ are each independently selected from the group consisting of substituted or unsubstituted alkyl groups, substituted or unsubstituted aryl groups, and substituted or unsubstituted aralkyl groups, R₁ to R₈ are each independently selected from the group consisting of a hydrogen atom, a deuterium atom, halogen atoms, substituted or unsubstituted alkyl groups, substituted or unsubstituted alkoxy groups, and substituted or unsubstituted aryl groups, and R₁₁ to R₁₄ are each independently selected from the group consisting of a hydrogen atom, substituted or unsubstituted alkyl groups, and substituted or unsubstituted aryl groups.

14. An electrochromic element comprising:
a pair of electrodes; and
an electrochromic layer disposed between the pair of electrodes,
wherein the electrochromic layer contains the organic compound as set forth in claim 1.

15. The electrochromic element according to claim 14,
wherein the electrochromic layer further contains a first organic compound different from the organic compound, and
wherein the first organic compound is selected from the group consisting of phenazine-based compounds, metallocene-based compounds, phenylenediamine-based compounds, and pyrazoline-based compounds.

16. The electrochromic element according to claim 14, wherein the electrochromic layer further contains an electrolyte.

17. The electrochromic element according to claim 14, wherein the electrochromic layer further contains a thickener.

18. An optical filter comprising:
the electrochromic element as set forth in claim 14; and
an active element connected to the electrochromic element.

19. A lens unit comprising:
the optical filter as set forth in claim 18; and
an imaging optical system including a lens.

20. An imaging device comprising:
the optical filter as set forth in claim 18; and
an imaging element configured to receive light passing through the optical filter.

21. A window comprising:
a pair of substrates;
the electrochromic element as set forth in claim 14, the electrochromic element being disposed between the pair of substrates; and
an active element connected to the electrochromic element.

22. An electrochromic mirror comprising:
the electrochromic element as set forth in claim 14;
an active element connected to the electrochromic element; and
a reflection member.
